(19) United States
(12) Patent Application Publication
(10) Pub. No.: US 2003/0232398 A1
(43) Pub. Date: Dec. 18, 2003

(54) Use of ROC plots of genetic risk factor to predict risk of sporadic breast cancer

(76) Inventors: James P. MacMurray, Loma Linda, CA (US); David E. Comings, Duarte, CA (US); Radhika Gade-Andavolu, Cathedral City, CA (US); Lawrence Cone, Palm Springs, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/401,132

(22) Filed: Mar. 28, 2003

Related U.S. Application Data
(60) Provisional application No. 60/367,747, filed on Mar. 28, 2002.

(51) Int. Cl. G01N 33/574

(52) U.S. Cl. 435/7.23

(57) ABSTRACT

The invention relates to methods for diagnosing a person's susceptibility for having an increased risk for the development of breast cancer. The invention relates further to methods for treating persons diagnosed for having increased risk for the development of said disease, in order to prevent the development of said disease.

### CROSS-REFERENCE TO RELATED APPLICATION

The present application is related to and claims priority under 35 U.S.C. § 119(e) to U.S. provisional patent application Serial No. 60/367,747, filed Mar. 28, 2002.

### FIELD OF THE INVENTION

The invention relates to methods for diagnosing or determining a person's susceptibility for having an increased risk for the development of breast cancer. The invention relates further to methods for treating persons diagnosed for having increased risk for the development of said disease, in order to prevent the development of said disease. The publications and other materials used herein to illuminate the background of the invention, and in particular, cases to provide additional details respecting the practice, are incorporated by reference and are further described in the attached bibliography.

### BACKGROUND OF THE INVENTION

Sporadic breast cancer is likely to be a polygenic disorder, involving multiple genes each accounting for only a small fraction of the variance with considerable genetic heterogeneity. While a number of genes have been shown in at least one study to be significantly associated with sporadic breast cancer, as is typical of polygenic disorders, there is considerable variability from study to study. We have proposed elsewhere that the most powerful method of identifying the genetic factors.disorders that are due to the additive effect of multiple genes is to examine the additive effect of multiple candidate genes (Comings et al. 2000a; Comings et al. 2000b). It is possible to compensate for the small effect size of each gene by examining the additive variance of multiple candidate genes. This also compensates for genetic heterogeneity since a number of different combinations of candidate genes can contribute to the total variance. When genes are scored such that the three genotypes are assigned a value of 0 to 2 depending upon their relative strength as risk factors, a total risk score can be formed by adding the scores for each individual gene. This risk score can be examined in a Receiver Operator Characteristic (ROC) plot of specificity versus sensitivity to determine whether it has sufficient power to be clinically useful (Zweig and Campbell, 1993; Metz, 1978).

Choice of candidate genes. A number of demographic risk factors have been identified for sporadic breast cancer. The most important of these relate to factors associated with the duration of exposure of the breast to estrogens. Estrogens increase the risk of breast cancer through various mechanisms and at various phases of life (Persson, 2000; Kahn et al., 1998). The over-expression of estrogen receptors or over-exposure of receptors to estrogen in normal breast epithelium augments the risk of breast cancer (Khan et al., 1998). Thus, factors that increase or prolong the exposure to estrogens tend to be risk factors for breast cancer. These are: age (Kelsey and Bernstein, 1996); early versus late age at menarche (Huang et al., 2000; Titus-Ernstoff, 1998; Negri et al., 1998; Kvale and Heuch, 1988); nulliparity (Tavani et al., 1999); late age of first full-term pregnancy (Kavani et al, 1999; Thompson and Janerich); breast feeding (Newcomb, 1997); increased interval between menarche and birth of the first child (Lavecchia et al., 1987; Brignone et al., 1987); years of education (which delays childbirth), number of years since last birth and oral contraceptive use (Tavani et al, 1999); later age at menopause (Paffenbarger et al, 1980; Fioretti et al., 1999); and postmenopausal hormone therapy (Colditz, 1997). The protection afforded by early full-term pregnancy in women could be explained by the higher degree of differentiation of the mammary gland at the time in which an etiologic agent or agents act. Cell proliferation is of importance for cancer initiation, whereas differentiation is a powerful inhibitor (Russo and Russo, 1999).

In general the odds ratios for these risk factors are in the range of 1.1 to 2.5. Those that tended to be associated with the highest odds ratios were the ones most likely to have a direct effect on duration of estrogen exposure such as age, age of onset of menarche, and years between the onset of menarche and the birth of the first child. Here the odds ratios could be as high as 5.0 or greater Tavani et al, 1999; Brignone et al., 1987). These same risk factors were also the ones most likely to hold for Hispanic and African American women (Mayberry and Branch, 1994; Mayberry and Stoddard-Wright, 1992; and Dupont et al., 1989). Early menarche is reported to be associated with raised estradiol levels persisting into early adult life (Stoll, 1998).

The early age of onset of menarche is one of the prominent risk factors for breast cancer and six candidate genes play a direct or indirect role in regulating the age of onset of menarche or other factors related to the duration of exposure to estrogen. These are leptin (LEP), leptin receptor (LEPR), catechol-o-metlhyl transferase (COMT), dopamine D₂ receptor gene (DRD2), estrogen receptor 1 gene (ESR1) and androgen receptor (AR) gene. Breast cancer is the most common form of cancer among women in Europe and America. Approximately 1 in 10 women in Western countries will develop breast cancer during their lifetime (Murphy, 1998). The autosomal dominant form of breast cancer, due to the presence of the BRCAI or BRCA2 genes, accounts for less than 5% of breast cancer cases in Caucasians (Carter, 2001). The remaining 95% are sporadic cases with only a modest or no family history of breast or ovarian cancer.

Leptin is a peptide hormone that has a role in the regulation of body weight, and has effects on metabolic, neuroendocrine, reproductive and immune systems (Ozet et al, 2001). It is the major regulator of the onset of menarche, a major risk factor in breast cancer. Plasma leptin levels have been shown to differ between breast cancer cases and controls (Petridou et al, 2000). Leptin is expressed in malignant epithelial cells of the breast (O'Brien et al, 1999) and the possible use of leptin as a chemical marker for breast cancer has been suggested (Tessitore et al, 2000). Association of variants of the LEP gene with breast cancer risk factors indicate a likely role for the association of variants of the leptin receptor (LEPR) with breast cancer risk as well.

Dopamine D₂ Receptor gene (DRD2). A significant association between the DRD2 gene and early onset of sexual activity has previously been observed (Miller et al., 1999). In addition, the dopamine D₂ receptor plays an important role in the regulation of prolactin, and prolactin has been implicated in breast cancer risk in a number of ways (Mandala et al., 1999; Llovera et al., 2000; and Goffin et al., 1999). Finally, dopamine is required for the action of leptin (Szcypka et al., 2000). Dopamine D₂ receptors are present on the surface of breast cancer cells (Sokoloff et al., 1989).

COMT is involved in the metabolic inactivation pathway for catechol estrogens (Lavigne et al, 2001). Also, in part owing to its ability to inhibit prolactin release, dopamine is thought to playa role in breast cancer pathogenesis (Johnson et al, 1995). High levels of prolactin suppress production of estrogen and progesterone, and these effects are blocked by dopamine agonists (Ibid). Striatal dopamine-stimulated adenylate cyclase activity appears to protect or inhibit mammary tumor development in rats (Goldman & Vogel, 1984), and dopamine 02 receptors are present in human breast cancer cell lines (Sokoloff et al, 1989).

Estrogen receptor 1 gene (ESR1). The estrogen receptor is a ligand mediated transcription factor. The ESR1 gene was chosen as a candidate gene because estrogen plays a central role in many of the hypotheses about the cause of breast cancer. Racial variations in the expression of ESR1 in normal breast tissue have been proposed to explain some of the racial differences in breast cancer (Lawson et al., 1999).

Androgen receptor gene (AR). The androgen receptor is also a ligand mediated transcription factor. The AR gene is located on the X-chromosome at Xq 11-12 (Brown et al., 1989). Two sets of polymorphic trinucleotide repeat sequences, CAG (Edwards et al., 1992) and GGC (GGN) (Sleddens et al., 1993), resulting in polyamino acid tracts in the protein, are present in the first exon of the AR gene. The shorter repeat alleles are associated with increased expression of the AR gene while longer repeats are associated with decreased expression (Chamberlain et al., 1994; Choong et al., 1996; Irvine et al., 1995; and Giovannucci et al., 1997). The S alleles of the AR gene are associated with an earlier age of onset of menarche (Comings et al., 2002a). Rebbeck et al. reported that the AR gene played a role in modifying the effect of the BRCA1 gene on the risk for breast cancer (Rebbeck et al., 1999).

### BRIEF SUMMARY OF THE INVENTION

The present invention provides a method for determining whether an individual has an increased risk for the development of breast cancer. According to one aspect, this invention provides a method for determining an individual's susceptibility for having an increased risk or determining whether an individual has an increased risk for the development of breast cancer, said method comprising detecting the presence in the individual of at least one polymorphic breast cancer associated allele of a breast cancer associated gene selected from the group consisting of: the leptin gene (LEP); the leptin receptor gene (LEPR); and the catechol-O-methyltransferase gene (COMT).

According to another aspect, the invention provides a method for determining an individual's susceptibility for having an increased risk for the development of breast cancer or determining whether an individual has an increased risk for the development of breast cancer, said method comprising detecting the presence in the individual of at least one polymorphic breast cancer associated allele of at least two breast cancer associated genes selected from the group consisting of: the leptin gene (LEP); the leptin receptor gene (LEPR); the D2 receptor gene; the catechol-O-methyltransferase gene (COMT); and the AR gene.

In one embodiment, the method for determining an individual's susceptibility for having an increased risk for the development of breast cancer or determining whether an individual has an increased risk for the development of breast cancer, comprises determining in said individual the additive risks from the LEP, LEPR, DRD2 and COMT risk alleles, wherein risk of development of breast cancer in said individual increases according to the number of risk alleles derived from these four contributing genotypes. The risk associated alleles for each gene can be determined from the risk of breast cancer associated with the presence of a given polymorphic allele of a breast cancer risk associated gene alone or the risk of breast cancer associated with the gene in combination with one or more other of breast cancer risk associated alleles of other breast cancer risk associated genes, as determined in an ROC plot. Breast cancer associated alleles and genes include alleles of genes that show an independent association with breast cancer or an additive association with breast cancer when examined with one or more other genes of the present invention.

In another embodiment, the method for determining an individual's susceptibility for having an increased risk for the development of breast cancer or determining whether an individual has an increased risk for the development of breast cancer comprises determining in said individual the additive risks from two or more of the LEP, LEPR, DRD2, COMT and AR breast cancer associated alleles, wherein risk of development of breast cancer in said individual increases according to the number of risk alleles derived from these five contributing genotypes. The risk alleles for each gene can be determined from the risk of breast cancer associated with the gene alone.

According to another aspect, this invention provides a method for determining the specificity, sensitivity and positive and negative likelihood risk of an individual developing breast cancer, the method comprising determining a breast cancer risk score for the individual in an ROC plot.

According to another aspect, this invention provides a method for treating a person, determined to have an increased risk for the development of breast cancer, for the prevention of developing said disease, comprising administering to said person an effective amount of an agent counteracting the influence of one or more alleles of said genes.

According to another aspect, this invention provides a method for screening an individual determined for having an increased or decreased risk for the development of breast cancer, for the prevention of developing said disease, comprising placing said person at high risk in an effective program for breast cancer screening, such as mammography, digital (electronic) mammography, breast MRI, or ductal levage, and comprising placing said individual at quite low risk into a less intensive screening program.

In one embodiment, the detection of the presence of one or more of said polymorphic genes comprises determining the presence of one or more polymorphisms selected from the group consisting of the leptin gene (LEP) dinucleotide repeat polymorphism D7S1875 in a sample from said individual, wherein risk of development of breast cancer in said individual is low when the LEP D7S1875 polymorphism is heterozygous for ≦210/≧212 bp alleles, risk is intermediate when the polymorphism is homozygous for ≦210/≦210 bp alleles, and risk is high when the polymorphism is homozygous for ≧212/≧212 bp alleles; the leptin receptor gene, exon 3 (LEPR3), wherein risk of development of breast cancer in said individual is low when the LEPR3 polymorphism is heterozygous for ≦158/≧160 bp alleles, risk is intermediate when the polymorphism is homozygous for ≧160/≧160 bp alleles, and risk is high when the polymorphism is homozygous for ≦158/≦158 bp alleles; the D2 receptor gene (DRD2) TaqI polymorphism wherein risk of development of breast cancer in said individual is low when the DRD2 polymorphism is genotype 2/2, risk is intermediate when the polymorphism is 1/2, and risk is high when the polymorphism is homozygous for 1 1; the catechol-O-methyltransferase gene (COMT) Val 108 Met substitution, wherein risk of development of breast cancer in said individual is low when the COMT polymorphism is genotype 1/2 or 2/2, and risk is high when the p6lymorphism is homozygous for 1/1; and the Androgen receptor gene (AR) polymorphic trinucleotide repeat sequences, CAG and GGC (GGN), wherein the risk of development of breast cancer is low when the trinucleotide repeat is LL and risk is high when the trinucleotide repeat is SS.

In another embodiment, the detection of the presence of one or more of said polymorphic genes comprises determining the presence of one or more polymorphisms selected from the group consisting of the LEP dinucleotide repeat polymorphism in a sample from said individual, wherein risk of development of breast cancer in said individual is high when the LEP genotype is LL and said risk is low in said individual when the LEP genotype is SL or SS; the LEPR tetranucleotide repeat polymorphism in a sample from said individual, wherein risk of development of breast cancer in said individual is high when the LEPR genotype is SS or LL and low when the genotype is SL; the D2 receptor gene (DRD2) TaqIpolymorphism wherein risk of development of breast cancer in said individual is low when the DRD2 polymorphism is genotype 2/2, is intermediate when the genotype is 1/2, and high when the genotype is 1/1; the catechol-O-methyltransferase gene (COMT) Val 108 Met substitution, wherein risk of development of breast cancer in said individual is low when the COMT polymorphism is genotype 1/2 or 2/2 and high when the genotype is 1/1; and the Androgen receptor gene (AR) polymorphic trinucleotide repeat sequences, CAG and GGC (GGN), wherein the risk of development of breast cancer is low when the trinucleotide repeat is LL or S/L and the risk is high when the trinucleotide repeat is SS. In a preferred embodiment, the LEP S polymorphic allele includes dinucleotide repeat alleles that are 207 base pairs or less in size and the LEP L polynorphic allele includes dinucleotide repeat alleles that are 208 base pairs or more in size.

### BRIEF DESCRIPTION OF THE DRAWINGS

FIG. 1 illustrates the additive effect of the LEP, LEPR, DRD2 and COMT risk alleles described in Examples 1-4 within a Receiver-Operator-Curve to indicate the sensitivity and specificity of the additive influence of these four polymorphisms on the risk of individuals for the development of breast cancer.

FIG. 2 illustrates the additive effect of the LEP, LEPR, DRD2, AR and COMT risk alleles described in Example 8 within a Receiver-Operator-Curve to indicate the sensitivity and specificity of the additive influence of these four polymorphisms on the risk of individuals for the development of breast cancer

FIG. 3 demonstrates the frequency of alleles for the LEP S and L alleles utilized in Example 8.

### DETAILED DESCRIPTION OF THE INVENTION

The first step in developing a multi-gene test for risk of a disease or disorder is to genotype candidate genes that are significantly associated with the disease or disorder. In exemplary embodiments, the present invention presents such methods wherein the disease or disorder is breast cancer. In these examples, candidate genes that each individually had been associated with breast cancer were chosen for further analysis. In analyzing genes suggestive of an association with breast cancer, all genes are scored 0, 1 or 2 depending upon whether a given genotype showed the least (0), intermediate (1), or strongest (2) association with breast cancer risk. Then a logistic regression analysis is performed on these data, with the dichotomous diagnosis score (controls=0, breast cancer=1) as the dependent variable and the gene scores as independent variables. Multivariate logistic regression analysis is used to determine which of the genes, in the presence of the other genes, continue to contribute to breast cancer risk. Next, a combined relative risk score for all of the genes discovered is determined based on adding together the scores of all of the genes selected by the logistic regression analysis.

The combined relative risk score is then analyzed in a Receiver Operator Characteristic (ROC) plot. A critical aspect of any test is to determine both its specificity and sensitivity. ROC curves plot the different values of the test against the specificity and 1-sensitivity of each value. ROC plots provide a pure index of the accuracy of a given test by demonstrating the limits of the tests ability to discriminate between alternative states of health or disease over the complete spectrum of operating conditions (Zweig & Campbell, 1993; These methods are also described in U.S. patent application Ser. No. 10/319,815). The ROC plot depicts the overlap between the two distributions by plotting the sensitivity versus 1-specificity for the complete range of decision thresholds. On the y-axis is sensitivity, or the true-positive fraction [defined as (number of true-positive test results)/(number of true-positive+number of false-positive test results)].

Once a set of candidate genes and alleles has been identified, markers for said genes and alleles can be utilized in diagnostic determination of an individual's breast cancer risk. The determination can be carried out either as a DNA analysis according to well known methods, which include indirect DNA sequencing of the normal and mutated genes at said genetic loci, allele specific amplification using the polymerase chain reaction (PCR) enabling detection of either normal or mutated sequences at said genetic loci, or by indirect detection of the normal or mutated alleles at said genetic loci by various molecular biology methods including e.g. PCR-single stranded conformation polymorphism (SSCP)-method or denaturing gradient gel electrophoresis (DGGE). Determination of the normal or mutated alleles at said genetic can also be done by single restriction fragment length polymorphism (RFLP)-method.

The determination can also be carried out at the level of RNA by analyzing RNA expressed at tissue level using various methods. Allele specific probes can be designed for hybridization. Hybridization can be done e.g. using Northern blot, Rnase protection assay or in situ hybridization methods. RNA derived from the normal or mutated alleles at said genetic loci can also be analyzed by converting tissue RNA first to cDNA and thereafter amplifying cDNA by an allele specific PCR-method and carrying out the analysis as for genomic DNA as mentioned above.

In some instances, the determination can also be carried out at the level of protein expression by analyzing a protein expressed by one or more of said alleles of said genes using various methods that are readily appreciated by those of ordinary skill in the art.

A person determined for having an increased risk for the development of breast cancer can be treated for the prevention of developing said diseases by administering to said subject an effective amount of an agent counteracting the influence of the mutated alleles at said genetic loci. This can be done by specific gene therapy aimed to repair the mutated sequences at said genetic loci, or by administering pharmacotherapies, which are aimed to modulate synthesis, release or metabolism of the endogenous gene products at said genetic loci, or to interact in a specific manner at said gene product target sites by modulating effects of the said gene products with specific receptor proteins specific to said gene products.

Influence of the mutated sequences at said genetic loci on the function of said genes can be investigated in transgenic animals. A transgenic animal can be generated using targeted homologous recombination methodology. Both normal and mutated sequences of said human genes (or any DNA sequence comprising a nucleotide sequence encoding said genes, or part thereof encoding the amino acid sequence of the mature mouse or a human mature copies of said genes, where either i) substitutions at said genes are unchanged or ii) have been substituted, will be introduced into the sequence of said genes to replace the endogenous signal peptide sequence. Under these conditions, the endogenous functions of said genes are otherwise normal, but the synthesis of the said genes is regulated by either normal or mutated human sequences of said genes. This transgenic model can be used to investigate in a very specific manner the physiological importance of the mutated copies of said genes. It will also provide an ideal preclinical model to investigate and screen new drug molecules, which are designed to modify the influence of the mutated versions of said genes.

Useful diagnostic techniques include, but are not limited to fluorescent in situ hybridization (FISH), direct DNA sequencing, PFGE analysis, Southern blot analysis, single stranded conformation analysis (SSCA), RNase protection assay, allele-specific oligonucleotide (ASO), nested PCR followed by restriction enzyme digestion, dot blot analysis and PCR-SSCP. Also useful are techniques employing DNA microchip technology.

Predisposition to breast cancer or risk of breast cancer can be ascertained by testing any tissue of a human for mutations of one or more candidate genes and/or alleles. Detecting the presence in an individual for one or more alleles can comprise analysis of a nucleic acid or a protein encoded by a nucleic acid, using techniques well known to those of ordinary skill in the art, including, but not limited to, detection of an allele in vivo, in situ, or ex vivo, by, e.g., removal of a protein or nucleic acid source (e.g., such as tissue or blood or other bodily fluid) from the individual and analysis of the source for the presence of the allele. By way of example, the presence of an allele can be determined by testing DNA from any tissue of the person's body, which will provide an indication of the presence of the allele in the individual. Most simply, blood can be drawn and DNA extracted from the cells of the blood. In addition, prenatal diagnosis can be accomplished by testing fetal cells, placental cells or amniotic cells for polymorphisms, e.g., in vivo, ex vivo and/or in situ.

There are several methods well known to persons of ordinary skill in the art that can be used to detect DNA sequence variations associated with polymorphisms, including e.g., direct DNA sequencing, clamped denaturing gel electrophoresis, heteroduplex analysis and chemical mismatch cleavage. An allele-specific detection approach such as allele-specific oligonucleotide (ASO) hybridization can be utilized to rapidly screen large numbers of other samples for candidate genes and/or alleles.

Detection of point mutations can be accomplished, e.g., by molecular cloning of the allele(s) and sequencing the allele(s) using techniques well known to persons of ordinary skill in the art. Alternatively, the gene sequences can be amplified directly from a genomic DNA preparation using known techniques. The DNA sequence of the amplified sequences then can be determined directly or with restriction enzyme analysis to detect polymorphic sites.

DNA sequences of a gene which have been amplified by use of PCR may also be screened using allele-specific oligomer probes, each of which contains a region of the gene sequence harboring a known mutation. For example, one oligomer may be about 30 nucleotides in length (although shorter and longer oligomers can be used, as recognized by those of ordinary skill in the art), corresponding to a portion of the gene sequence. By use of a battery of such allele-specific probes, PCR amplification products can be screened to identify the presence in an individual of an allele. Hybridization of allele-specific probes with nucleic acids amplified from cells can be performed, for example, on a nylon filter. Hybridization to a particular probe under high stringency hybridization conditions indicates the presence of the same mutation in the cells as in the allele-specific probe.

Nucleic acid analysis via microchip technology is also applicable to the present invention. In this technique, literally thousands of distinct oligonucleotide probes can be applied in an array on a silicon chip.- A nucleic acid to be analyzed is fluorescently labeled and hybridized to the probes on the chip. It is also possible to study nucleic acid- protein interactions using these nucleic acid microchips. Using this technique one can determine the presence of mutations, sequence the nucleic acid being analyzed, or measure expression levels of a gene of interest. The method is one of parallel processing of many, even thousands, of probes at once and can tremendously increase the rate of analysis.

Alteration of mRNA transcription can be detected by any techniques known to persons of ordinary skill in the art. These include by way of example Northern blot analysis, PCR amplification and RNase protection. Diminished mRNA transcription can indicate an alteration of the wild-type gene.

Polymorphisms in a gene can also in some instances be detected by screening for alteration of the protein encoded by the gene. For example, monoclonal antibodies immunoreactive with an allele can be used to screen a tissue. Lack of cognate antigen would indicate absence of an allele. Antibodies specific for products of an allele also could be used to detect the product of the allele. Such immunological assays can be done in any convenient format known in the art. These include Western blots, immunohistochemical assays and ELISA assays. Any means for detecting an altered protein can be used to detect polymorphisms of gene. Functional assays, such as protein binding determinations, also can be used. In addition, assays which detect biochemical function can be used.

The diagnostic method of the present invention is useful to clinicians for aiding decisions as to an appropriate course(s) of treatment. It is also contemplated by the present invention that determination of heterozygosity versus homozygosity will further aid in diagnosis or prognosis of breast cancer or breast cancer risk.

Primer pairs specific for a gene or allele are useful for determination of the nucleotide sequence of a particular allele using PCR. The pairs of single-stranded DNA primers can be annealed to sequences within or surrounding a gene in order to prime amplifying DNA synthesis of the gene itself. Allele-specific primers also can be used. Such primers anneal only to particular alleles of interest, and thus will only amplify a product in the presence of the particular allele as a template. In one embodiment, the allele-specific primers will amplify a nucleic acid comprising a particular allele but not other allelic variants.

In order to facilitate subsequent cloning of amplified sequences, primers may have restriction enzyme site sequences appended to their 5′ ends. Thus, all nucleotides of the primers are derived from sequences specific for a gene or sequences adjacent to the gene, except for the few nucleotides necessary to form a restriction enzyme site. Such enzymes and sites are well known to persons of ordinary skill in the art. The primers themselves can be synthesized using techniques that are well known to persons of ordinary skill in the art. Generally, the primers can be made using oligonucleotide synthesizing machines that are commercially available.

The nucleic acid probes provided by the present invention are useful for a number of purposes. They can be used, e.g., in Southern hybridization to genomic DNA and in the RNase protection method for detecting point mutations. The probes can be used to detect PCR amplification products. They may also be used to detect mismatches with a gene or mRNA using other techniques well known in the art.

In order to detect a gene variant, a biological sample is prepared and analyzed for a difference between the sequence of the allele being analyzed and the sequence of other known alleles. In a preferred embodiment, the disease or disorder is breast cancer or a risk of developing breast cancer and the polymorphism detected is one or more polymorphisms in the group of genes consisting of the LEP, LEPR, D2, AR and COMT.

“Antibodies.” The present invention also provides for detection of polymorphic specific alleles with the use of polyclonal and/or monoclonal antibodies and fragments thereof, and immunologic binding equivalents thereof, which are capable of specifically binding to a polypeptide or specific nucleotide sequence and fragments thereof that are indicative of the presence of an allele associated with breast cancer or the risk of breast cancer. The term antibody is used both to refer to a homogeneous molecular entity, or a mixture such as a serum product made up of a plurality of different molecular entities. Antibodies will be useful in assays as well as pharmaceuticals. Antibodies to an allele will particularly be useful in detecting the allele and aiding in the diagnosis of a predisposition to breast cancer.

An immunological response is usually assayed with an immunoassay. Normally, such immunoassays involve some purification of a source of antigen, for example, that produced by the same cells and in the same fashion as the antigen. A variety of immunoassay methods are well known by persons of ordinary skill in the art.

As used herein, the singular form “a”, “an”, and “the” include plural references unless the context clearly indicates otherwise.

As used herein, the terms “diagnosing” or “prognosing,” as used in the context of breast cancer or breast cancer risk, are used to indicate classification, severity or monitoring of the disease progression, prior to, during or after treatment.

Polynucleotide compositions useful in the practice of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule (Peptide Nucleic Acids or “PNA's”). The polynucleotides of the invention may be isolated or substantially pure. Oligonucleotides which detect the genes utilized in the present invention or analogues of such oligonucleotides can also be prepared. Such analogues may constitute alternative structures such as “PNA's” or the like. It is evident that these alternative structures, representing the sequences of the present invention, are likewise part of the present invention.

cDNA or genomic libraries of various types may be screened as natural sources of the nucleic acids of a particular allele, or such nucleic acids may be provided by amplification of sequences resident in genomic DNA or other natural sources, e.g., by PCR. The choice of cDNA libraries normally corresponds to a tissue source which is abundant in mRNA for the desired proteins. Phage libraries are normally preferred, but other types of libraries may be used. Clones of a library are spread onto plates, transferred to a substrate for screening, denatured and probed for the presence of desired sequences.

Polynucleotide polymorphisms associated with particular alleles from candidate genes for breast cancer or breast cancer risk can be detected by hybridization with a polynucleotide probe which forms a stable hybrid with that of the target sequence, under highly stringent to moderately stringent hybridization and wash conditions. If it is expected that the probes will be perfectly complementary to the target sequence, high stringency conditions will be used. As well known by those of ordinary skill in the art, hybridization stringency may be lessened if some mismatching is expected, for example, if variants are expected with the result that the probe will not be completely complementary. Conditions are chosen which rule out nonspecific/adventitious bindings, that is, which minimize noise.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. The stringency conditions are dependent on the length of the nucleic acid and the base composition of the nucleic acid, and can be determined by techniques well known by persons of ordinary skill in the art.

The probes can include an isolated polynucleotide attached to a label or reporter molecule and may be used to isolate other polynucleotide sequences having sequence similarity, by standard methods. Other similar polynucleotides may be selected by using homologous polynucleotides. Alternatively, polynucleotides encoding these or similar polypeptides may be synthesized or selected by use of the redundancy in the genetic code. Various codon substitutions may be introduced, e.g., by silent changes (thereby producing various restriction sites) or to optimize expression for a particular system. Mutations may be introduced to modify the properties of the polypeptide, perhaps to change ligand-binding affinities, interchain affinities, or the polypeptide degradation or turnover rate.

Polypeptides comprising a particular allele, if soluble, may be coupled to a solid-phase support, e.g., nitrocellulose, nylon, column packing materials (e.g., Sepharose beads), magnetic beads, glass wool, plastic, metal, polymer gels, cells, or other substrates. Such supports may take the form, for example, of beads, wells, dipsticks, or membranes.

“Recombinant nucleic acid” is a nucleic acid which is not naturally occurring, or which is made by the artificial combination of two otherwise separated segments of sequence. This artificial combination is often accomplished by either chemical synthesis means, or by the artificial manipulation of isolated segments of nucleic acids, e.g., by genetic engineering techniques.

In order to detect the presence of a group of alleles predisposing an individual to breast cancer or breast cancer risk, a biological sample such as blood is prepared and analyzed for the presence or absence of predisposing alleles by analyzing the individual's genetic material. As used herein, the analysis of genetic material may be direct, through examination of a nucleic acid, or indirect, such as by examination of an altered amino acid produced by the individual's genetic material. Such diagnoses may be performed by diagnostic laboratories, or, alternatively, diagnostic kits are manufactured and sold to health care providers or to private individuals for self-diagnosis.

The practice of the present invention employs, unless otherwise indicated, conventional techniques of chemistry, molecular biology, microbiology, recombinant DNA, genetics, immunology, cell biology, cell culture and transgenic biology, which are within the skill of the art.

Preferred embodiments relating to methods for detecting polymorphisms include enzyme linked immunosorbent assays (ELISA), radioimmunoassays (RIA), immunoradiometric assays (IRMA) and immunoenzymatic assays (IEMA), including sandwich assays using monoclonal and/or polyclonal antibodies.

General Methods

Study samples Controls: Selected as a control sample for Examples 1-7 were a group of older non-Hispanic Caucasian University students. Subjects were MAST—screened to exclude alcoholism, generating a sample of 165 participants comprised of 81 males and 84 females with an age range of 21 to 49, mean age 34.3. Selected as a control sample for Example 8, the controls were 145 sex, geographical area, and race matched subjects from the Loma Linda University Public Health Clinics. The mean age of the controls in Example 8 was 43.0 years (S.D. 12.96). The range was 23 to 66 years.

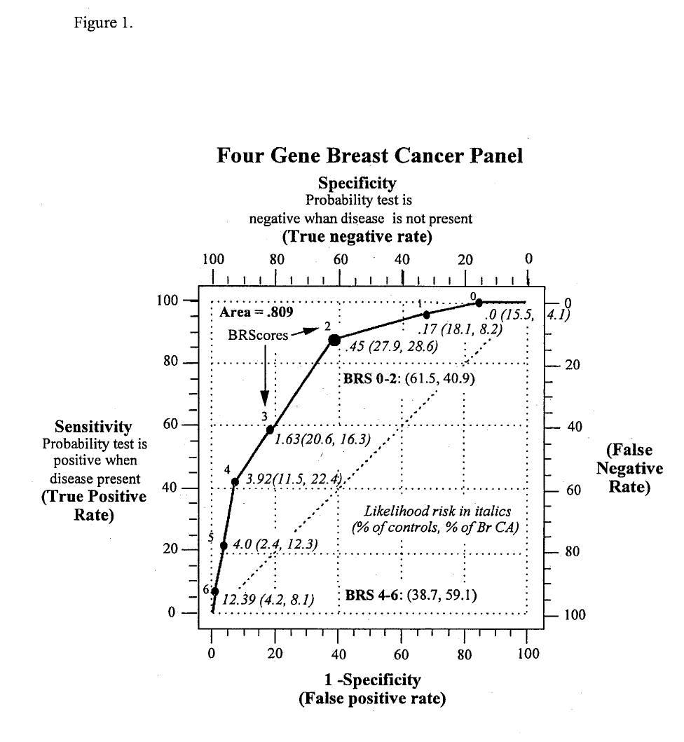

Cases in Examples 1-4, FIG. 1 and Tables 1 and 2: Forty-nine non-Hispanic Caucasian women, ages 30-85, mean age 63.3, with a history of breast cancer were studied. The age at diagnosis of breast cancer ranged from 30 to 84, with a mean age at diagnosis of 62.4. All subjects had been treated by either quadrantectomy or mastectomy. All but two also received either adjuvant radiation, hormonal or chemotherapy. Blood samples were obtained from all subjects, and DNA was extracted using traditional means. Written informed consent was obtained from all subjects.

Statistical methods. Alleles and genotype distributions were determined between breast cancer and controls. The Chi-square (χ²) test was employed to statistically compare these groups. All statistical data calculations were done with the SPSS statistical package for Macintosh (release 6.1.1) (SPSS, Inc, Chicago, Ill.).

The practice of the present invention employs, unless otherwise indicated, conventional techniques of chemistry, molecular biology, microbiology, recombinant DNA, genetics, immunology, cell biology, cell culture and transgenic biology, which are within the skill of persons of ordinary skill in the art.

The present invention is described by reference to the following Examples, which are offered by way of illustration and are not intended to limit the invention in any manner. Standard techniques well known by persons of ordinary skill in the art and/or the techniques specifically described and or referenced herein were utilized.

### EXAMPLES

### Example 1

Analysis of association of LEP gene with breast cancer risk. Initial studies of leptin were largely focused on the ability of this hormone to control fat metabolism (Tessitore et al, 2000). However, it was subsequently discovered that this hormone also exerts important regulatory influence over a host of other biological domains, ranging from timing of puberty (Urbanski, 2001) to mediation of immune function (Faggioni et al, 2001; Zarkesh-Esfahani et al, 2001; Sanchez-Margalet & Martin-Romero, 2001). Leptin is a 16 kDa protein that has been shown to play a role in innate and acquired immunity (Faggioni et al, 2001). Leptin has a direct effect on the generation of an inflammatory response, via activation of leukocytes (Zarkesh-Esfahani et al, 2001). The leptin receptor is expressed in monocytes as well as in CD4(+) and CD8(+) T lymphocytes, where it has been shown to have the signaling capacity to activate JAK-STAT cascade (Sanchez-Margalet & Martin-Romero, 2001).

Leptin is the product of the ob gene. In 1994 Friedman and colleagues (1996) cloned and sequenced the mouse ob gene and its human OB homologue. These studies demonstrated that the ob gene encoded a 4.5-kb mRNA that was expressed in adipose tissue. The dinucleotide repeats present on the YAC contig containing the human OB gene described by Green et al, 1995, and now referred to as LEP. Among the contigs, D7S1875 was closest to the LEP gene, and was therefore chosen for study. The D7S1875 is a dinucleotide repeat polymorphism whose alleles range in size from 198 to 224 bp in length. There are two distinct allele ‘groups’, with the lowest bp (198 and 200 bp) comprising over 50% of the allelic distribution in normal controls, and a second common allele group beginning at 212 bp. Therefore, using the most commonly employed method of analysis for dinucleotide repeat polymorphisms, we segregated the repeats into short vs. long alleles with ‘short’ repeats comprising those ≦210 bp, and ‘long’ repeats comprising those ≧212 bp. This method was then used to create three ‘genotypes’: homozygosity for short repeats (≦210/≦210 bp), heterozygosity (≦210≧212 bp), and homozygosity for long repeats (≧212/≧212 bp). Example 1 is based on a study of the association of the LEP dinucleotide repeat polymorphism in a sample of 165 normal control subjects and 49 female breast cancer cases. All subjects were non-Hispanic Caucasians. The control subjects (81 males and 84 females) had an age range of 21 to 49 with a mean age of 34.3. The Breast cancer cases had an age range of 30 to 85, with a mean age of 63.3 and a mean age at diagnosis of 62.4. All cases had been treated by either quadrantectomy or mastectomy. The frequency of homozygosity for the long bp repeats was 18% in controls and 43% in breast cancer (BRCA) cases. The frequency of heterozygosity was 51% in controls and 25% in breast cancer cases. Group analysis, by Chi-Square, revealed a Pearson p=0.0003. Example 1 indicates that heterozygosity at the LEP 1875 polymorphism is associated with decreased risk for development of breast cancer, whereas homozygosity for the long repeats is associated with increased risk for the development of breast cancer.

LEP protocol: The accession number for the leptin (LEP) gene D7S1875 polymorphism is AC018662. DNA was prepared by standard procedures from whole-blood samples. Specific primers were used based on a computer assisted search, and these are shown below: SEQ ID NO: 1: 5′ GCCTAAGGGAATGAGACACA 3′ forward primer; and SEQ ID NO: 2: 5′ ATGTGAGTTTGCCAAGAGCT 3′. The single stranded dry DNA was dissolved in 10 mM Tris and 1 mM EDTA. The forward primer was labeled with fluorescent HEX at the 5′ end at the last coupling cycle in DNA synthesis. A fluorescently labeled primer that anneals to one strand of the target DNA was used during PCR to label specific regions of DNA for the subsequent steps. A standard concentration was achieved by optical density on a Perkin-Elmer UV Spectrophotometer. Polymerase chain reaction (PCR) was performed on the DNA of the subjects. The solution for the amplification of each gel of 36 samples of LEP was 460.8 ul of deionized water, 60 ul of 10× buffer, 12 ul of dinucleotriphosphates, 12 ul of each primer, and 3.12 ul of Taq polymerase. The reagents for the PCR reaction were taken from a commercial kit (Qiagen, Inc, 1997). Reaction volume was 15 ul containing 20 ng DNA. 1.8 ul deionized formamide, 0.3 ul of the GENESCAN-500 Rox, and 0.2 ul of loading dye were spun along with 0.5 ul of PCR products. Samples were run on an ABI-373 using GENESCAN and GENOTYPER software.

### Example 2

Analysis of association of LEPR with breast cancer risk. The leptin receptor gene (LEPR) is located on chromosome 1 p in humans (Chung et al, 1996a). The LEPR has at least five splice variants in mice (Lee et al, 1996), and the genomic structure of the human leptin receptor and identification of two novel intronic micro satellites has been described by Chung et al (1996b). One of these micro satellites is described as the human leptin receptor (LEPR) gene, exon 3 (ibid), accession #U59248. The methods for genetic analysis of LEPR are described by Thompson et al, 1996. A single common repeat, at 158 bp, characterizes the distribution of LEPR repeats in normal samples. Therefore, we segregated the repeats into short vs. long alleles with ‘short’ repeats comprising those ≦158 bp, and ‘long’ repeats comprising those ≧160 bp. This method was then used to create three ‘genotypes’: homozygosity for short repeats (≦158/≦158 bp), heterozygosity (≦158/≧160 bp), and homozygosity for long repeats (≧160≧160 bp). The present invention is based on a study of the association of the LEPR dinucleotide repeat polymorphism in a sample of 165 normal control subjects and 49 female breast cancer cases. All subjects were non-Hispanic Caucasians. The control subjects (81 males and 84 females) had an age range of 21 to 49 with a mean age of 34.3. The Breast cancer cases had an age range of 30 to 85, with a mean age of 63.3 and a mean age at diagnosis of 62.4. All cases had been treated by either quadrantectomy or mastectomy. The frequency of homozygosity for the shortbp repeats (≦158/≦158 bp), was 33% in controls and 63% in breast cancer cases. The frequency of heterozygosity was 55% in controls and 29% in BRCA cases. Group analysis, by Chi-Square, revealed a Pearson p=0.0008. The present study indicates that heterozygosity at the LEP R polymorphism is associated with decreased risk for development of BRCA risk, whereas homozygosity for the short repeats (≦158/≦158 bp) is associated with increased risk for the development of breast cancer.

LEPR protocol: The accession number for the leptin receptor gene exon 3 polymorphism is #U59248. DNA was prepared by standard procedures from whole-blood samples. Specific primers were used based on a computer assisted search, and these are shown below:

| | |
|---|---|
| 5′CCTTCCCAACCTCCTAAAGACAACCTG 3′ | (SEQ ID NO:3) |
| 5′TGTACAGATCTGTGCTATTTTTGCAGC 3′ | (SEQ ID NO:4) |

Single stranded dry DNA was dissolved in 10 mM Tris and 1 mM EDTA. The forward primer was labeled with fluorescent Amidite (FAM) at the 5′ end at the last coupling cycle in DNA synthesis. A fluorescently labeled primer that anneals to one strand of the target DNA was used during PCR to label specific regions of DNA for the subsequent steps. A standard concentration was achieved by optical density on a Perkin-Elmer UV Spectrophotometer.

Polymerase chain reaction (PCR) was performed on the DNA of the subjects. The solution for the amplification of each gel of 36 samples of LEPR3 was 460.8 μl of deionized water, 60 μl of 10× buffer, 12 μl of dinucleotriphosphates, 12 μl of each primer, and 3.12 μl of Taq polymerase. The reagents for the PCR reaction were taken from a commercial kit (Qiagen, Inc, 1997). Reaction volume was 15 μl containing 20 ng DNA. 1.8 μl deionized formamide, 0.3 μl of the GENESCAN-500 Rox, and 0.2 μl of loading dye were spun along with 0.5 ul of PCR products. Samples were run on an ABI-373 using GENESCAN and GENOTYPER software.

### Example 3

Analysis of association of D2 with breast cancer risk. Dopamine interacts with estrogenic activity and immune responses in several ways that suggest its potential role in the mediation of breast cancer risk. Dopaipinergic input to the ventromedial hypothalamus is known to facilitate the estrogen-induced luteinizing hormone surge in ewes (Anderson et al, 2001). Anterior pituitary dopamine has been shown to play an important role in estrogen-induced anterior pituitary hyperplasia and tumor formation (Nedvidkova et al, 2001). Additional studies have suggested that dopamine has a novel role in regulating malignant cell proliferation and controlling immune functions in tumor-bearing animals (Basu & Dasgupta, 2000). Similarly, in part owing to its ability to inhibit prolactin release, dopamine is thought to playa specific role in breast cancer pathogenesis (Johnson et al, 1995). High levels of prolactin suppress production of estrogen and progesterone, and these effects are blocked by dopamine agonists (Ibid). Striatal dopamine-stimulated adenylate cyclase activity appears to protect or inhibit mammary tumor development in rats (Goldman & Vogel, 1984), and dopamine D2 receptors are present in human breast cancer cell lines (Sokoloff et al, 1989). The present invention is based on a study of the association of the DRD2 TaqI polymorphism in a sample of 165 normal control subjects and 49 female breast cancer cases. All subjects were non-Hispanic Caucasians. The control subjects (81 males and 84 females) had an age range of 21 to 49 with a mean age of 34.3. The breast cancer cases had an age range of 30 to 85, with a mean age of 63.3 and a mean age at diagnosis of 62.4. All cases had been treated by either quadrantectomy or mastectomy. The frequency of homozygosity for the DRD2 1/1 genotype was 2% in controls and 8% in breast cancer cases; the frequency of the 1/2 genotype was 33% in controls and 43% in breast cancer cases, and the frequency of homozygosity for the 2/2 genotype was 66% in controls and 49% in breast cancer cases. Group analysis, by Chi-Square, revealed a Pearson p=0.025. The present study indicates that the risk for development of breast cancer risk is low in individuals who are homozygous for the DRD2 TaqI A2 allele, whereas breast cancer risk increases as a linear function with the presence of the DRD2 TaqI A1 allele.

DRD2 protocol: The accession number for the DRD2 TaqI site is # AFOSO737. DNA was prepared by standard procedures from whole-blood samples. The DRD2 TaqI A (Grandy et al, 1989) bi-allelic polymorphism was determined using the conditions described. Allele nomenclature: “11”=homozygous for the 310 bp allele; “12”=heterozygous 310/180 bp alleles; “22”=homozygous for the 180 bp allele.

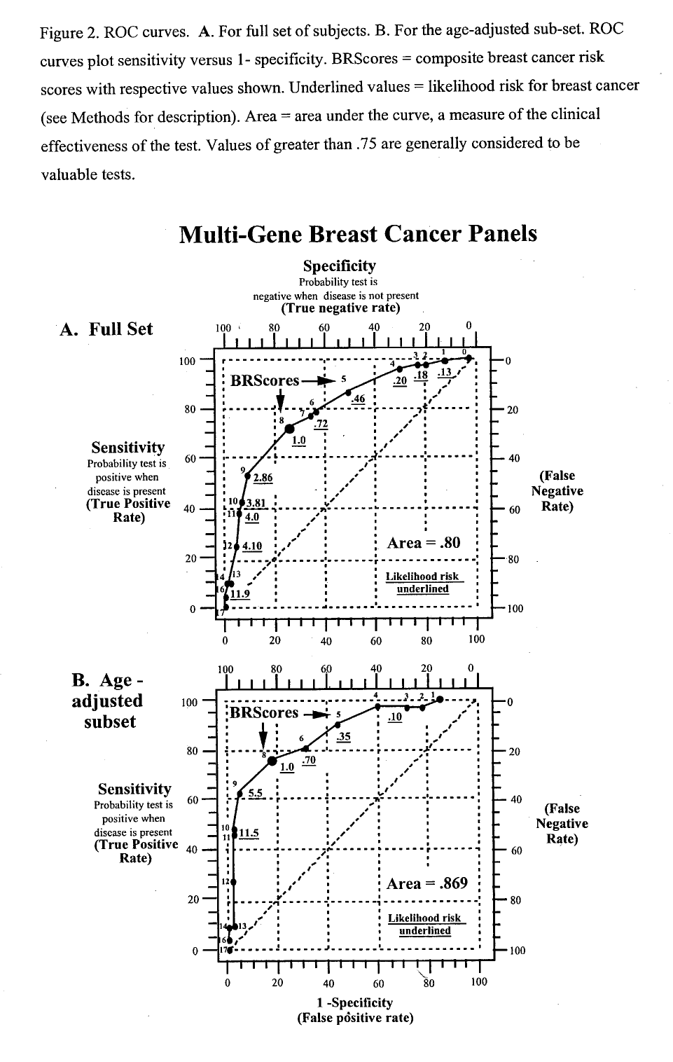

In Example 8, the same allele analysis of DRD2 was performed on a breast cancer population of N=67. The results are depicted in FIG. 2 and Tables 3-5.

### Example 4

Analysis of association of COMT gene with breast cancer risk. O-methylation catalyzed by catechol-O-methyltransferase (COMT) is a Phase II metabolic inactivation pathway for catechol estrogens (Lavigne et al, 2001). COMT catalyzes the methylation of catechol estrogens to methoxy estrogens, which simultaneously lowers the potential for DNA damage and increases the concentration of 2-methoxyestradiol, an antiproliferative metabolite (Dawling et al, 2001). Thus, inherited alterations in COMT catalytic activity may contribute to interindividual differences in breast cancer risk associated with estrogen-mediated carcinogenicity. A 3- to 4-fold decreased methylation activity of COMT has been linked to a G to A transition in the COMT gene, differentiating the COMT-1 and COMT-2 alleles (Lachman et al, 1996). Several studies have examined the association between the COMT G to A polymorphism and breast cancer, yielding conflicting results (Huang et al, 1999; Lavigne et al, 1997, 2001; Millikan et al, 1998), with widely ranging associations tending to vary as a function of premenopausal vs. postmenopausal diagnoses. The present invention is based inter alia on an analysis of the association of the COMT polymorphism in a sample of 165 normal control subjects and 49 female breast cancer cases. All subjects were non-Hispanic Caucasians. The control subjects (81 males and 84 females) had an age range of 21 to 49 with a mean age of 34.3. The Breast cancer cases had an age range of 30 to 85, with a mean age of 63.3 and a mean age at diagnosis of 62.4. All cases had been treated by either quadrantectomy or mastectomy. The frequency of homozygosity for the COMT 1 allele (e.g., 1/1 genotype) was 19% in controls and 52% in breast cancer cases. There was no difference in genotype frequencies between groups for either the 1/2 or 2/2 genotypes. Group analysis, by Chi-Square, revealed a Pearson p=0.0004. The present study indicates that homozygosity for the 1/1 genotype at the COMT G to A transition polymorphism is associated with increased risk for development of breast cancer risk. Results of this analysis are further described in FIG. 1 and Tables 1 and 2.

COMT protocol: The COMT G to A transition polymorphism is 474 bp from the start site, producing a Val 108 Met substitution in the soluble form (at position 1949 in Accession #Z26491). DNA was prepared by standard procedures from whole-blood samples. The PCR-based assay was similar to that described by Li et al (1997), and the genotyping methods were similar to those described by Mitrunen et al (2001). The 217-bp PCR products were amplified using specific primers (SEQ ID NO: 5: 5′ TCG TGG ACG CCG TGA TTC AGG-3′; and SEQ ID NO: 6: 5′ AGG TCT GAC AAC GGG TCA GGC-3′).

The resulting amplified products were digested using the NlaIII enzyme (New England Biolabs, Beverly, Mass.). The presence of an additional cleavage site differentiated the variant COMT-2 allele from the wild-type COMT-1 allele.

In Example 8, the same allele analysis of COMT was performed on a breast cancer population of N=67. The results are depicted in FIG. 2 and Tables 3-5.

### Example 5

Analysis of association of AR gene with breast cancer risk. As described above for the AR gene the GGN tri-nucleotide repeat in exon 1 was used (Sleddens et al., 1993). The alleles were divided into ≦16 repeats (S) and ≧17 repeats (L). The SS, SL and LL genotypes were examined.

### Example 6

Analysis ofassociation of ER gene with breast cancer risk. For the ESR1 gene, the Xba I polymorphism was used (Kobayishi et al., ). In studies of osteoporosis show an association with the 12 heterozygotes, i.e., positive heterosis (Comings, 2000(c)).

The DNA sequence of the mutant alleles or said gene products associated with any other cleavage product of LEP, LEPR, DRD2, COMT or AR genetic loci can be used for screening a subject to determine if said subject is a carrier of breast cancer risk alleles at these genetic loci.

### Example 7

Table 1, below, shows the genotypes of controls and breast cancer cases for four genetic polymorphisms as described in Examples 1, 2, 3 and 4 described of the present invention.

TABLE 1
Comparison of Breast Cancer Cases and Controls at Four
Polymorphic Loci as described in Examples 1, 2, 3 and 4: LEP,
LEPR, DRD2, and COMT.

| Polymorphism | | LEP | | N | Pearson |
|---|---|---|---|---|---|
| Genotype | ≦210/≦210 | ≦210/≧212 | ≧212/≧212 | | |
| Controls | 52 (32%) | 84 (51%) | 29 (18%) | 165 | |
| Breast Cancer | 16 (33%) | 12 (25%) | 21 (43%) | 49 | P = 0.0003 |
| Polymorphism | | LEPR | | | |
| Genotype | ≦158/≦158 | ≦158/≧160 | ≧160/≧160 | | |
| Controls | 55 (33%) | 91 (55%) | 19 (12%) | 165 | |
| Breast Cancer | 31 (63%) | 14 (29%) | 4 (8%) | 49 | P = 0.0008 |
| Polymorphism | | DRD2 | | | |
| Genotype | 1/1 | ½ | 2/2 | | |
| Controls | 3 (2%0 | 54 (33%) | 108 (66%) | 165 | |
| Breast Cancer | 4 (8%) | 21 (43%) | 24 (49%) | 49 | P = .025 |
| Polymorphism | | COMT | | | |
| Genotype | 1/1 | ½ | 2/2 | | |
| Controls | 32 (19%) | 84 (51%) | 49 (30%) | 165 | |
| Breast Cancer | 26 (53%) | 14 (29%) | 9 (18%) | 49 | P = .00002 |

Each gene was codified at each of the four polymorphisms by their contribution to low vs. high breast cancer risk. Those genotypes conferring a low risk were coded as 0; those conferring an intermediate risk were coded as 1; and those conferring a high risk were coded as 2. Then a logistic regression analysis was performed on these data, with the dichotomous diagnosis score (controls=0, breast cancer=1) as the dependent variable and the gene scores as independent variables. Table 2 shows the results of this logistic regression analysis.

TABLE 2
Logistic Regression Analysis

| | B | SE B | R | R² | P |
|---|---|---|---|---|---|
| DRD2 | .7517 | .32382 | .1213 | .015 | .0203 |
| LEP | .8678 | .2369 | .2227 | .049 | .0002 |
| LEPR | .7141 | .2089 | .2051 | .042 | .0006 |
| COMT | .6520 | .1946 | .2001 | .040 | .0008 |
| (Constant) | −3.570 | .4906 | | | <.0001 |

Sum R² = .146

These results showed that each of the genes, in the presence of the effect of all of the other genes, was independently contributing to breast cancer risk, that three of these genes (LEP, LEPR, COMT) were individually significant at p<0.001, that each gene individually contributed to between 1.5 and 4.9% of the variance of breast cancer, and that combined they contributed to 14.6% of the variance.

The cumulative codes for each subject, both controls and breast cancer cases, were then evaluated using a ROC plot. ROC plots provide a pure index of the accuracy of a given test by demonstrating the limits of the tests ability to discriminate between alternative states of health or disease over the complete spectrum of operating conditions (Zweig & Campbell, 1993). The ROC plot depicts the overlap between the two distributions by plotting the sensitivity versus 1-specificity for the complete range of decision thresholds. On the y-axis is sensitivity, or the true-positive fraction [defined as (number of true-positive test results)/(number of true-positive+number of false-positive test results)]. This has also been referred to as positivity in the presence of a disease based on calculations for the affected group. On the x-axis is the false-positive fraction, or 1-specificity [defined as (number of false-positive results)/(number of true-negative+number of false-positive results)]. This is an index of specificity and is calculated from the unaffected group (Zweig & Campbell, 1993). Thus, sensitivity=true-positive results/total subjects with the disease and specificity =true-negative results/total subjects without the disease.

Computer programs considerably enhance the ease of use of ROC curves (Zweig & Campbell, 1993). These allow the determination of the positive and negative likelihood ratios for the presence of disease for each of the sensitivity-specificity pairs. For the positive likelihood ratios those with the lowest risk=1 and those with a higher risk show progressively higher values of the test score.

For the negative likelihood ratio, those with the highest risk=1 and those with progressively lower values of the test score. The product of the two, termed here the likelihood risk, is useful since those who a have neutral risk have scores of approximately 1, those with a diminished risk have scores less than 1, and those with a higher risk have scores of greater than 1. The program also calculates the area under the curve, a further measure of the effectiveness of the test. The use of a ROC curve using the additive risk for LEP, LEPR, DRD2, and COMT alleles as described in Examples 1, 2, 3 and 4 for an estimate of breast cancer risk, is shown in FIG. 1.

### Example 8

Analysis of the association of 5 different polymorphisms with breast cancer risk was also performed. Additional breast cancer individuals were identified and compared to the controls as described. To obtain a population based rather than a referral based sample of breast cancer, the cases were ascertained from the private practice of oncologists in the Rancho Mirage, California area. An additional eighteen breast cancer cases were added to the 49 cases described in previous Examples. The majority of the breast cancer women were postmenopausal and did not have a strong family history of breast cancer. DNA was isolated using standard techniques at the Genetic Research Institute of the Desert for PCR analysis on certain genes. Aliquots of DNA was transferred to the Department of Medical Genetics at the City of Hope Medical Center for more genotyping.

The mean age of the breast cancer subjects was 69.0 years (S.D. 12.54. The range was 30 to 96 years. The mean age of the controls was 43.0 years (S.D. 12.96). The range was 23 to 66 years. To examine the issue of whether the difference in age between the breast cancer cases and controls was a factor, we selected a subset of each group. The division of cases was dictated by attempting to produce equal number of cases in each group, still have adequate power, and utilize the older of the controls and the younger of the breast cancer cases. Thus, for this division, only the controls that were 51 years of age or older and only breast cancer cases that were 75 years of age or younger were used. This produced a set of 45 controls with an average age of 58.3 years (S.D. 4.7) and 43 breast cancer cases with an average age of 63.4 years (S.D. 10.6). This is termed the ‘age-adjusted subset.’

In 1996 an association between the D7S1875 polymorphism of the LEP gene and obesity was reported in young females (Comings et al., 1996). The distribution of the alleles at the D7S11875 dinucleotide repeat demonstrated two major peaks with the shorter alleles (S) ranging from 199 to 207 bp in length, and the longer alleles (L) ranging from 208 to 225 bp in length. Studies indicated that the different lengths of microsatellite polymorphisms play a role in gene regulation (Comings et al. 1998). These studies led to an examination of the association of the human LEP gene with age of onset of menarche in females. This showed a significant three way interaction between LEP genotypes, age of menarche and maternal age, i.e., age of the mothers when the probands were born(Comings et al., 2001). This showed that the S/S LEP genotype was associated with a low age of menarche in women with a maternal age of ≧30 years, while the L/L LEP genotype was associated with a low age of menarche in those with a maternal age of <30 years. Analysis of breast cancer risk associated with this LEP allele distinction are shown in Tables 3-5 and FIG. 2.

For the LEPR gene we used a tetranucleotide (CTTT) repeat polymorphism Pacak et al.; Chung et al., 1997). There was a range from 1 to 9 repeats. The alleles were divided into those with 4 of fewer repeats (S) versus 5 or more (L). The division was based on optimizing the similarity in the size of the two groups. Analysis of breast cancer risk associated with this LEPR allele distinction are shown in Tables 3-5 and FIG. 2.

Chi square analyses. The results for the Chi Square analyses of each of the genes for breast cancer subjects versus controls for the alleles described in Examples 3, 4 and 8 and the breast cancer samples described in‘Example 8 are shown in Table 3A-F. For the LEP gene (Table 3A) there was a significant (p≦0.00013) increase in the frequency of the LL genotype and decrease in the frequency of SL heterozygotes in subjects with breast cancer compared to controls. For the LEPR gene there was a significant (p≦0.0005) increase in frequency of the SS genotype, a decrease in the frequency of the SL genotype and an increase in the frequency of the LL genotype showing a negative heterosis

For the COMT gene in the larger breast cancer sample of N=67, there was a significant (p≦0.0004) increase in the frequency of the more highly expressed 1 or G allele showing an increase in the frequency of the 11 genotype and a decrease in the frequency of the 12 and 22 genotypes in breast cancer.

There was a modest but not significant increase in the frequency of the 11 and 12 genotypes of the DRD2 gene in breast cancer in the larger breast cancer sample of N=67. For the AR gene, there was a significant (≦0.014) increase in the SS genotype and a decrease in the frequency of the remaining two genotypes of the GGN repeat polymorphism in breast cancer in the larger breast cancer sample of N=67. This genotype is associated with increase activity of the AR gene. By contrast there was no significant association of the ESR1 gene with breast cancer.

ANOVA, gene coding and regression analysis. For the following analyses a breast cancer contribution variable was made in which the controls were scored as 0 and the breast cancer cases as 1 for each gene. Breast cancer contribution was used as the dependent variable in ANOVA to determine the mean score for each genotype in the larger breast cancer sample of N=67. Thus, the higher the score the more the genotype is associated with breast cancer. Based on these results, each genotype of each gene was scored as 0, 1 or 2 depending upon its relative breast cancer contribution score. Those with the lowest mean breast cancer contribution score were scored 0, those with the highest mean were scored 2, and the remaining genotype was scored 0 or 2 depending upon whether it was closer to the 0 or 2 mean, and 1 if it was clearly intermediate. These were termed the gene scores. By our convention, the 11 genotype is listed first, the 12 genotype second and the 22 genotype third. Thus, if the highest mean breast cancer contribution scores were associated with the 11 genotype, intermediate scores with the 12 genotype and the lowest scores with the 22 genotype, the gene score would be 210.

To determine the percent of the variance of breast cancer attributable to a given gene, regression univariate regression analysis was used with breast cancer contribution as the dependent variable and the gene score as the independent variable. This produced r, the correlation coefficient, and r² the fraction of the variance attributable to that gene, and p, the significance level. Table 4A-F shows the results of the ANOVA test with the F ratio and p value, the resulting gene code, r², and the p values for the r² in the larger breast cancer sample of N=67. The asterisks represent the results of a post hoc Tukey test of the ANOVA, highlighting those means that were significantly different at alpha ≦0.05. In addition, we have added the r² and p value results for the age-adjusted subset. Because of the smaller number of cases, this subset has considerably less power than the full set. Thus, the critical result was the r² value, rather than the p value, to determine if the fraction of the variance was dramatically decreased in this sample.

The ANOVA for the LEP gene allele described in Example 8 was significant (p≦0.0001). The means were highest for the LL genotype and intermediate for the SS genotype giving a gene code of 102 in the larger breast cancer sample of N=67. It accounted for 7.3% of the variance for the full set (p≦0.0001). This dropped only slightly, 6.1%, for the age-adjusted subset. The ANOVA for the LEPR gene allele described in Example 8 was significant (p≦0.0004). The 12 heterozygotes had the lowest mean breast cancer contribution scores while the mean for the 11 and 22 genotypes were similar giving a 202 gene score. This gene accounted for 6.4% of the variance in the full set (p≦0.0002). This increased to 10.3% in the age-adjusted subset. The ANOVA for the COMT gene was significant (p≦0.0003), with the highest mean for the 11 genotype and lowest for the 12 and 22 genotype giving a gene score of 200 in the larger breast cancer sample of N=67,. It accounted for 7.3% of the variance. This increased to 18.8% in the age-adjusted subset (p≦0.0001). As with the Chi square, the ANOVA was also not significant for the DRD2 gene (p 0.13) in the larger breast cancer sample of N=67. The association with the genotypes was 1 allele co-dominant producing a gene score of 210. This accounted for 1.8% of the variance and this was just significant (p≦0.05). The percent of the variance increased to 2.8% in the age-adjusted subset. The ANOVA for the AR gene was significant (p≦0.014). The association was S allele codominant producing a gene score of 210. This accounted for 4.0% of the variance (p≦0.0035). This increased to 9.9% in the age-adjusted subset. The ANOVA for the ESR1 gene was not significant (p≦0.39). The mean breast cancer contribution scores were highest for the 11 and 12 genotypes producing a gene score of 220. This accounted for only 0.9% of the variance (p≦0.17). This decreased to 0.4% for the age-adjusted subset (p≦0.55).

The results in Example 8 clearly indicated that the difference in mean ages between the controls and breast cancer cases in the full set in the larger breast cancer sample of N=67, did not explain the positive results. Except for the ESR1 gene, the percent of the variance increased for each gene in the age-adjusted subset in the larger breast cancer sample of N=67.

Multivariate regression analysis for the alleles of Example 8. Using the breast cancer contribution variable as the dependent variable and each gene code as independent variables, a multivariate regression analysis was performed using SPSS. The results for both the full set and the age-adjusted subset, are shown in Table 5A and B. For the full set (Table A), all the genes except the ESR1 gene were included in the equation. The genes are sorted by T value. All were significant at p≦0.014. The percent of the variance ranged from 7.8% for the LEP gene to 2.3% for the DRD2 gene. The total explained variance was 24%. The adjusted value was 22%.

The results for the age-adjusted set are shown in Table 5B, again sorted by T value. As for the full set, the ESR1 gene was excluded from the equation. Even though it was significant when examined individually, the AR gene was also excluded from the multivariate analysis. The variances for the remaining genes ranged from 17.0% for the COMT gene, 13.4% for the LEPR gene, 10.1% for the LEP gene and 2.1% for the DRD2 gene. The total variance explained by all four genes was 40.1%, the adjusted r² was 0.372.

The statistical program used for these studies was multivariate regression analysis (SPSS). However, since the dependent variable was dichotomous we also performed multivariate logistic regression analysis (SAS). There results were essentially the same. The multivariate regression analysis (SPSS) program produced Beta or r, and thus r², for each gene. When the multivariate logistic regression program (SAS) was used it was necessary to progressively calculate each r² value by sequential subtraction.

Breast cancer risk and ROC plots for the alleles of Example 8. For a given individual, the breast cancer risk consisted of the sum of the gene scores for each gene. For the full set, only the ESR1 gene was excluded. Breast cancer risk varied from 0 (2 cases) to 17 (2 cases). The mean was 7.13, S.D. 3.90, and median 6.0. It approximated a normal distribution with a skewness of 0.31 and kurtosis of −0.43. The breast cancer risk for the total set of alleles and frequencies described for the alleles of Example 8 and the larger breast cancer sample of N=67 was evaluated in a ROC plot (FIG. 2A). The area under the curve was 0.80. The likelihood risks ranged from 0.13 for those with a breast cancer risk score of 1, to 11.9 for those with a score of 16. Since calculation of the likelihood risks for breast cancer risk scores of 0 and 17 would involve multiplying or dividing by 0, these could not be calculated.

For the age-adjusted subset, the breast cancer risk score varied from 1 (7 cases) to 17 (2 cases). The ESR1 gene was excluded. The mean was 7.80, S.D. 4.1 and median of 8.0. It again approximated a normal distribution with a skewness of 0.14 and kurtosis of −0.68. The BCRS for the age-adjusted subset is shown in FIG. 2B. The area under the curve was 0.869. The likelihood risk ranged from 0.10 for those with a BCRS of 1 through 4, to 11.5 for those with a breast cancer risk score of 10 through 12. It then dropped for scores of 13 through 17. This was likely to be due to the fact that these scores were fairly rare and subject to fluctuation.

TABLE 3
Chi square analyses for each gene of the alleles of Examples 8
and the larger breast cancer sample of N = 67.

| A. LEP gene N (%) | | | | | | |
|---|---|---|---|---|---|---|
| Group | N | SS | SL | LL | X² | p |
| Breast Cancer | 67 | 21 (31.3) | 20 (29.9) | 26 (38.8) | | |
| Controls | 145 | 52 (35.9) | 73 (50.3) | 20 (13.8) | 17.87 | .00013 |
| B. LEPR gene N (%) | | | | | | |
| Group | N | SS | SL | LL | X² | p |
| Breast Cancer | 67 | 34 (50.7) | 21 (31.3) | 12 (17.9) | | |
| Controls | 145 | 50 (34.2) | 85 (58.2) | 10 (6.9) | 15.23 | .0005 |
| C. COMT gene N (%) | | | | | | |
| Group | N | 11 | 12 | 22 | X² | p |
| Breast Cancer | 67 | 31 (46.3) | 24 (35.8) | 12 (17.9) | | |
| Controls | 145 | 29 (20.0) | 78 (53.8) | 38 (26.2) | 15.59 | .0004 |
| D. DRD2 gene N (%) | | | | | | |
| Group | N | 11 | 12 | 22 | X² | p |
| Breast Cancer | 67 | 5 (7.5) | 26 (38.8) | 36 (53.7) | | |
| Controls | 145 | 4 (2.8) | 46 (31.7) | 95 (65.5) | 4.09 | .130 |
| E. AR gene N (%) | | | | | | |
| Group | N | SS | SL | LL | X² | p |
| Breast Cancer | 67 | 41 (61.2) | 22 (32.8) | 4 (6.0) | | |
| Controls | 145 | 61 (42.1) | 59 (40.7) | 25 (17.2) | 8.47 | .014 |
| F. ESR1 gene N (%) | | | | | | |
| Group | N | 11 | 12 | 22 | X² | p |
| Breast Cancer | 67 | 10 (14.9) | 35 (52.2) | 22 (32.8) | | |
| Controls | 145 | 19 (13.1) | 64 (44.1) | 62 (42.8) | 1.84 | .39 |

TABLE 4
ANOVA, Gene scores and r² for each gene of the alleles of
Example 8 and the larger breast cancer sample of N = 67.

| | | | | | | Gene | | |
|---|---|---|---|---|---|---|---|---|
| Genotype | N | Mean | S.D. | F | p | score | r² | p |
| A. LEP gene | | | | | | | | |
| SS | 73 | .29* | .46 | | | | | |
| SL | 93 | .21* | .41 | | | | | |
| LL | 46 | .56 | .50 | 9.62 | .0001 | 102 | .073 | .0001 |
| Age-adjusted subset | | | | | | | .061 | .0195 |
| B. LEPR | | | | | | | | |
| gene | | | | | | | | |
| SS | 84 | .40* | .49 | | | | | |
| SL | 106 | .20 | .40 | | | | | |
| LL | 22 | .55* | .51 | 8.09 | .0004 | 202 | .064 | .0002 |
| Age-adjusted subset | | | | | | | .103 | .0023 |
| C. COMT | | | | | | | | |
| gene | | | | | | | | |
| 11 | 60 | .52 | .50 | | | | | |
| 12 | 102 | .24* | .43 | | | | | |
| 22 | 50 | .24* | .43 | 8.29 | .0003 | 200 | .073 | .0001 |
| Age-adjusted subset | | | | | | | .188 | ≦.0001 |
| D. DR2 gene | | | | | | | | |
| 11 | 9 | .55 | .53 | | | | | |
| 12 | 72 | .36 | .48 | | | | | |
| 22 | 131 | .27 | .44 | 2.06 | .13 | 210 | .018 | .05 |
| Age-adjusted subset | | | | | | | .028 | .12 |
| E. AR gene | | | | | | | | |
| SS | 102 | .40 | .49 | | | | | |
| SL | 81 | .27 | .45 | | | | | |
| LL | 29 | .14* | .35 | 4.35 | .014 | 210 | .040 | .0035 |
| Age-adjusted subset | | | | | | | .099 | .003 |
| F. ESR1 | | | | | | | | |
| gene | | | | | | | | |
| 11 | 29 | .34 | .48 | | | | | |
| 12 | 99 | .35 | .48 | | | | | |
| 22 | 84 | .26 | .44 | .94 | .39 | 220 | .009 | .171 |

| | | |
|---|---|---|
| Age-adjusted subset | .004 | .551 |

Significantly different from highest (or lowest) value at p ≦ .05 by Tukey test.

TABLE 5
Multivariate regression analysis of the alleles of Example 8 and the larger
breast cancer sample of N = 67.
A. Full Sample*

| Gene | r | r² | T | P |
|---|---|---|---|---|
| LEP | .280 | .078 | 4.59 | <.0001 |
| LEPR | .237 | .056 | 3.86 | .0002 |
| COMT | .188 | .035 | 3.01 | .0029 |
| AR | .163 | .027 | 2.64 | .0089 |
| DRD2 | .151 | .023 | 2.48 | .0139 |
| Total | .490 | .240# | 13.01(F) | <.0001 |

#adjusted r² = .221
*ESR1 gene was excluded from the equation by the regression analysis.

B. Age-adjusted Subset**

| Gene | r | r² | T | P |
|---|---|---|---|---|
| COMT | .413 | .170 | 4.84 | <.0001 |
| LEPR | .367 | .134 | 4.26 | .0001 |
| LEP | .318 | .101 | 3.70 | .0004 |
| DRD2 | .145 | .021 | 1.69 | .0934 |
| Total | .633 | .401# | 13.91(F) | <.0001 |

#adjusted r² = .372
•ESR1 and AR genes were excluded from the equation by the
regression analysis.

The present invention incorporates a number of unique aspects and features for the investigation of the molecular genetics of breast cancer.

An emphasis on sporadic breast cancer. While the identification of single genes that are causative of familial breast cancer is an exciting development, the vast majority of breast cancer is sporadic with a minimal or negative family history and polygenically inherited rather than due to single genes.

Emphasis on genes for known breast cancer risk factors. Since other reports of sporadic breast cancer have also examined genes for estrogen metabolism, emphasis on the study of genes related to demographic risk factors is not unique. However, the emphasis on genes especially associated with age of menarche and related variables, is unique.

Examining the additive effect of multiple genes. The major characteristic of polygenic disorders is that each gene contributed to a small percent of the total variance. As a result, variation from study to study is the expected outcome (Comings DE. 2002). Since polygenic disorders are due to the additive effect of multiple genes, they are best studied by examining the additive effect of multiple genes (Comings et al. 2000a). This approach provides added power and helps to diminish some of the variability from study to study. Thus, the total explained variance for the fives genes used in Example 8 was 24% for the full set, and 40% for the age-adjusted set. Both were highly significant. If all five genes shown in Example 8 to have an additive affect are included, even if the total variance varies considerably from study to study, it is likely that the total variance would be significant for all studies.

Test for r² rather than significance. While significance levels are commonly used in the studies of the genetics of complex disease, a far more important parameter is effect size, which can be measured by the calculation of r². In the full set of the alleles of Example 8 larger breast cancer sample of N=67 analyzed by multivariate regression analysis, the r² for the LEP and LEPR genes of 0.078 and 0.056 respectively were high, and when only the age-adjusted set was examined, the r² values for these two and the COMT genes of 0.170 to 0.101 were even higher. These results compare favorably with previous studies showing r values of 0.005 to 0.03 (Comings et al., 2000a; Comings et al., 2000b). There is a great deal of genetic heterogeneity in polygenic disorders. Thus, using the DRD2 gene as an example, it might show a significant association (by itself) for one group of subjects, but a non-significant association in another group. The advantage of using multiple genes to form a total risk score and a ROC plot is that it helps to compensate for this heterogeneity. If is it not important for one group, it does not contribute, but by not eliminating it from a set, if it is important in another group, it is there. Only the genes that contribute very little to the total variance are eliminated. In summary, it is important to think in terms of r² (variance) rather than p values. Thus all sets of data given are valid.

Production of the breast cancer risk score. The individual gene scores can be added to produce a composite summary gene score for the entire set. This has the advantage that it provides a single variable whose magnitude is a measure of the number of risk alleles that each woman has inherited. In general, the higher the score the higher the risk.

ROC plot of the breast cancer risk score. An estimate of the relative likelihood risk for breast cancer for each value of a breast cancer risk score, the assessment of the sensitivity and specificity of each value, and the total area under the ROC curve provide useful analytical tools for breast cancer risk assessment. The results of the ROC plot of the breast cancer risk score for the alleles of Example 8 indicated that five genes could be of considerable clinical usefulness in assessing a woman's risk for sporadic breast cancer (FIG. 2). The likelihood risks varied from 0.13 to 11.9 for the full set and 0.10 to 11.5 for the age-adjusted subset. Thus, individual women varied over a 90 to 115 fold range in their risk for breast cancer.

Increased variance for age-adjusted subset. Despite the smaller number of subjects in the age-adjusted subset, the r² values for the alleles of Example 8 were higher for the LEP, LEPR and COMT genes, and the total variance of 0.401 was considerably higher than the total of 0.24 for the full set. Without being bound by theory, this perhaps may be a reflection of the elimination of the breast cancer women older than 75 years of age. This would tend to eliminate those women for whom non-genetic factors such as age per se were the major risk factors, and increase the relative proportion of younger but still primarily postmenopausal women in whom the genetic factors we identified may be more important as risk factors.

The present invention demonstrates how the genotypes at several breast cancer risk genes can be combined into a breast cancer risk score and how this can be evaluated in a ROC plot to produce a useful guide for a given women about her risk for breast cancer. Related genes may also be important and can be included in the practice of this invention. The identification of a number of polymorphisms on a single chip is now possible. Use of the ROC curves could thus provide a simple and low cost test to identify a woman's risk for postmenopausal sporadic breast cancer. This would be of considerable benefit in allowing the most efficient use of screening and preventive procedures

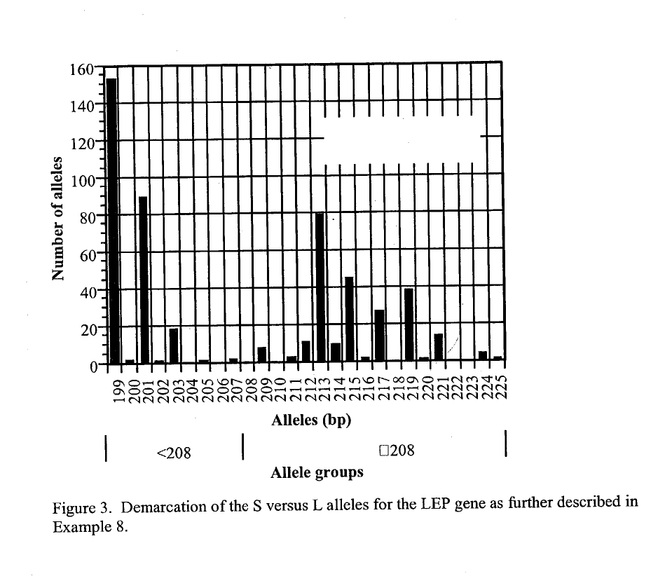

The division of the LEP alleles into S and L groups can be presented slightly differently in different embodiments of the. invention but, as shown in FIG. 3, these differences are minor and do not impact the overall nature of the invention. In the preferred embodiment, the invention is practiced by selecting 207 base pairs as the upper range of the S allele for LEP and 208 as the lower range for the L allele, as in Example 8. It would make no difference to the nature of the present invention, however, if the cut point was a 204,205, 206, 207, 206,209, 210, or 211 base pairs.

### REFERENCES

Anderson ST, Walsh JP, Tillet Y, et al. Dopaminergic input to. the ventromedial hypothalamus facilitates the oestrogen-induced luteinizing hormone surge in ewes. 2001 Neuroendocrinology 73(2):91-101.

Basu S, Dasgupta PS. Role of dopamine in malignant tumor growth. 2000 Endocrine 12(3):237-41.

Brignone G, Cusimano R, Dardanoni G, Gugliuzza M, Lanzarone F, Scibilia V, and Dardanoni L. A case-control study on breast cancer risk factors in a southern European population. Int J Epidemiol 1987;16:356-61.

Brown C J, Goss S J, Lubahan D B, Joseph D R, Wilson E M, French F S et al. Androgen receptor locus on the human X chromosome: regional localization to Xq11-12 and description of a DNA polymorphism. Am. J Hum. Genet. 1989;44:264-9.

Carter RF. BRCA1, BRCA2 and breast cancer: a concise clinical review. 2001 Clin Invest Med 24(3):147-57.

Chamberlain N L, Driver E D and Miesdeld R L. The length and location of CAG trinucleotide repeats in the androgen receptor N-terminal domain affect transactivation function. Nucleic Acids Res. 1994;22:3181-6.

Choong CS, Kemppainen J A, Zhou A-X and Wilson E M. Reduced androngen receptor gene expression with first exon CAG repeat expansion. Molec.Endocr. 1996;10:1527-35.

Chung W K, Power-Kehoe L, Chua M, et al. Genomic structure of the human OB receptor and identification of two novel intronic microsatellites. 1996a Genome Res 6: 1192-99.

Chung W K, Power-Kehoe L, Chua M, et al. Mapping of the OB receptor (OBR) to 1 p in a region of non-conserved gene order from mouse and rat to human. 1996b Genome Res 6:431-8.

Chung W K, Power-Kehoe L, Chua M, Chu F, Aronne L, Huma Z et al. Exonic and intronic sequence variation in the human leptin receptor gene (LEPR). Diabetes 1997;46:1509-11.

Colditz G A. Hormone replacement therapy increases the risk of breast cancer. Ann N Y Acad Sci 1997;833:129-36.

Comings D E, MacMurray J P, Gade R, Muhleman D and Peters W R. Genetic variants of the human obesity gene: Association with psychiatric symptoms and body mass index in young women, and interaction with the dopamine D2 receptor gene. Molecular Psychiatry 1996;1:325-35.

Comings D E. Polygenic inheritance and micro/minisatellites. Molecular Psychiatry, Jan. 3, 1998 (1):21-31.

Comings D E, Gade-Andavolu R, Gonzalez N, Wu S, Muhleman D, Blake H et al. Comparison of the role of dopamine, serotonin, and noradrenaline genes in ADHD, ODD and conduct disorder: multivariate regression analysis of 20 genes. Clin Genet 2000 (a);57:178-96.

Comings D E, Gade-Andavolu R, Gonzalez N, Wu S, Muhleman D, Blake H et al. A multivariate analysis of 59 candidate genes in personality traits: the temperament and character inventory. Clin Genet 2000 (b) ;58:375-85.

Comings D E and MacMurray J M. Molecular Heterosis: A Review. Molecular Genetics and Metabolism 2000(c);71:19-31.

Comings D E and MacMurray J. Maternal age as a confounding variable in association studies. Am. J Medical Genetics Neuropsychiatric Genetics 2001;105:564.

Comings D E, Muhleman D, Johnson J P and MacMurray J P. Parent-Daughter transmission of the Androgen Receptor (AR) gene as an explanation of the effect of father absence on age of menarche. Child Development 2002a;73:1046-51.

Comings D E. The Real Problem with Association Studies. Am. J Med. Genet. (Neuropsychiatric Genetics) 2002b;(in press).

Dawling S, Roodi N, Mernaugh R L, et al. Catechol-O-methyltransferase (COMT)-mediated metabolism of catechol estrogens: comparison of wild-type and variant COMT isoforms. 2001 Cancer Res 61(18):6716-22.

Dupont W D, Page D L, Rogers L W, and Parl F F. Influence of exogenous estrogens, proliferative breast disease, and other variables on breast cancer risk. Cancer 1989;63 :948-57.

Edwards A, Hammond H A, Jin L, Caskey C T and Chakraborty R. Genetic variation at five trimeric and tetrameric tandem repeat loci in four human population groups. Genomics 1992;12:241-53.

Faggioni R, Feingold K R, Grunfeld C. Leptin regulation of the immune response and the immunodeficiency of malnutrition. 200115(14):2565-71.

Fioretti F, Tavani A, Bosetti C, La Vecchia C, Negri E, Barbone F, Talamini R, and Franceschi S. Risk factors for breast cancer in nulliparous women. Br J Cancer 1999;79: 1923-8.

Friedman J M, Leibel R L, Siegel D S, et al., Molecular mapping of the mouse ob mutation. 1991 Genomics 11: 1 054-62.

Giovannucci E, Stampfer M J, Krithivas K, Brown M, Brufsky A, Hennekens C H et al. The CAG repeat within the androgen receptor gene and its relationship to prostate cancer. Proc.Natl.Acad.Sci. U.S.A. 1997;94:3320-3.

Goldman P R, Vogel W H. Striatal dopamine-stimulated adenylate cyclase activity reflects susceptibility of rats to 7,12-dimethylbenz(a)anthracene-induced mammary tumor development. 1984 Carcinogenesis 5(7):971-3.

Goffin V, Touraine P, Pichard C, Bernichtein S, and Kelly P A. Should prolactin be reconsidered as a therapeutic target in human breast cancer? Mol Cell Endocrinol 1999;151:79-87.

Grandy D K, Litt M, Allen L., et al. The human dopamine D2 receptor gene is located on chromosome 11 at q22-q23 and identifies a TaqI RFLP. 1989 Am J Hum Genet 45 :778-85.

Green E D, Maffei M, Braden V V, et al. The human obese (OB) gene: RNA expression pattern and mapping on the physical, cytogenetic, and genetic maps of chromosome 7. 1995 Genome Res 5:5-12.

Huang W Y, Newman B, Millikan R C, Schell M J, Hulka B S, and Moorman P G. Hormone-related factors and risk of breast cancer in relation to estrogen receptor and progesterone receptor status. Am J Epidemiol 2000;151:703-14.

Irvine R A, Yu M C, Ross R K and Coetzee G A. The CAG and GGC microsatellites of the androgen receptor gene are in linkage disequilibrium in men with prostate cancer. Cancer.Res. 1995;55:1937-40.

Johnson D E, Ochieng J, Evans S L. The growth inhibitory properties of a dopamine agonist (SKF 38393) on MCF-7 cells. 1995 Anticancer drugs 6(3):471-4.

Jonsson E, Sedvall G, Brene S, et al. dopamine-Related genes and their relationships to monoamine metabolites in CSF. 1996 Biol Psychiat 40:1.032-43.

Khan S A, Rogers M A, Khurana K K, Meguid M M and Numann P J. Estrogen receptor expression in benign breast epithelium and breast cancer risk. J Natl Cancer Inst 1998;90:37-42.

Kelsey J L and Bernstein L. Epidemiology and prevention of breast cancer.AnnuRevPublic Health 1996;17:47-67.

Kobayashi S, Inoue S, Hosoi T, Ouchi Y, Shiraki M and Orimo H. Association of bone mineral density with polymorphism of estrogen receptor gene. J Bone Miner.Res. 1996; 1:306-11.

Kvale G and Heuch I. Menstrual factors and breast cancer risk. Cancer 1988;62:1625-31.

Lachman H M, Papolos D F, Saito T, et al. Human catechol-O-methyltransferase pharmacogenetics: description of a functional polymorphism and its potential application to neuropsychiatric disorders. 1996 Pharmacogenetics 6:243-50.

La Vecchia C, Decarli A, Parazzini F, Gentile A, Negri E, Cecchetti G, and Franceschi S. General epidemiology of breast cancer in northern Italy. Int J Epidemiol 1987;16:347-55.

Lavigne J A, Goodman J E, Fonong T, et al. The effects of catechol-O-methyltransferase inhibition on estrogen metabolite and oxidative DNA damage levels in estradiol-treated MCF-7 cells. 2001 Cancer Res 61(20):7488-94.

Lawson J S, Field A S, Champion S, Tran D, Ishikura H and Trichopoulos D. Low oestrogen receptor alpha expression in normal breast tissue underlies low breast cancer incidence in Japan. Lancet 1999;354:1787-8.

Lee G H, Proenca R, Montez J M, et al. Abnormal splicing of the leptin receptor in diabetic mice. 1996 Nature 379(6566):632-5.

Li T, Vallada H, Curtis D, et al. Catechol-O-methyltransferase Val158Met polymorphism: frequency analysis in Ran Chinese subjects and allelic association of the low activity allele with bipolar affective disorder. 1997 Pharmacogenetics 7:349-53.

Lichtenstein P, Holm N V, Verkasalo P K, et al. Environmental and heritable factors in the causation of cancer-analyses of cohorts of twins from Sweden, Denmark, and Finland. 2000 N Engle J Med 343(2):78-85.

Llovera M, Touraine P, Kelly P A, and Goffin V. Involvement of prolactin in breast cancer: redefining the molecular targets. Exp Gerontol 2000;35:41-51.

Mandala M, Lissoni P, Ardizzoia A, Barni S, Rovelli F, Confalonieri G, Malugani F, Moro C, Fumagalli G, Giani L, and Tancini G. Endocrinological study of the dopaminergic regulation of prolactin release in metastatic breast cancer. Tumori 1999;85:494-7.Mayberry R M and Branch P T. Breast cancer risk factors among Hispanic women. Ethn Dis 1994;4:41-6.

Mayberry R M and Stoddard-Wright C. Breast cancer risk factors among black women and white women: similarities and differences. Am J Epidemiol 1992;136:1445-56.

Metz C E. Basic principles of ROC analysis. Seminars in Nuclear Medicine 1978;8:283-98.

Miller W B, Pasta D J, MacMurray J, Chiu C, Wu H and Comings DE. Dopamine receptor genes are associated with age at first intercourse. J Biosocial Science 1999;31:43-54.

Mitrunen K, Jourenkova N, Kataja V, et al. Polymorphic catechol-O-methyltransferase gene and breast cancer risk. 2001 Cancer epidemiol Biomarkers Prev 10(6):635-40.

Murphy M J jr. Molecular action and clinical relevance of Aromatase inhibitors. 1998 Oncologist 3(2): 129-30.

Negri E, La Vecchia C, Bruzzi P, Dardanoni G, Decarli A, Palli D, Parazzini F, and Rosselli del Turco M. Risk factors for breast cancer: pooled results from three Italian case-control studies. Am J Epidemiol 1988;128:1207-15.

Nedvidkova J, Pacak K, Haluzik M et al. The role of dopamine in methylene blue-mediated inhibition of estradiol benzoate-induced anterior pituitary hyperplasia in rats. 2001 Neurosci Lett 304(3): 194-8.

Newcomb P A. Lactation and breast cancer risk. J Mammary Gland Biol Neoplasia 1997;2:311-8.

O'brien S. N., Welter B. H., Price T M. Presence of leptin in breast cell lines and breast tumors. 1999 Biochem Biophys Res Commun 259(3):695-8.

Ozet A, Arpaci F, Yilmaz M. I., et al. Effects of tamoxifen on the serum leptin revel in patients with breast cancer. 2001 Jpn J Clin Onco 131 (9):424-7.

Pacak K, Palkovits M, Kopin I. J. and Goldstein D S. Stress-induced norepinephrine release in the hypothalamic paraventricular nucleus and pituitary-adrenocortical and sympathoadrenal activity: In vivo microdialysis studies. Fr.Neuroendocr. 1995;16:89-150.

Paffenbarger R. S. Jr, Kampert J. B., and Chang H. G. Characteristics that predict risk of breast cancer before and after the menopause. Am J Epidemiol 1980;112:258-68.

Petridou E, Papadiamantis Y, Markopoulos C, et al. Leptin and insulin growth factor I in relation to breast cancer (Greece). 2000 Cancer Causes Control 11(5):383-8.

Persson I. Estrogens in the causation of breast, endometrial and ovarian cancers—evidence and hypotheses from epidemiological findings. 2000 J Steroid biochem Mol Biol 74(5):357-64.

Rebbeck T R, Kantoff P W, Krithivas K, Neuhausen S, Blackwood M A, Godwin A K, Daly M B, Narod S A, Garber J E, Lynch H T, Weber B L, and Brown M. Modification of BRCA 1-associated breast cancer risk by the polymorphic androgen-receptor CAG repeat. Am J Hum Genet 1999;64:1371-7.

Risch H A, McLaughlin J R, Cole D E, et al. Prevalence and penetrance of germline BRCA1 and BRCA2 mutations in a population series of 649 women with ovarian cancer. 2001 Am J Hum Genet 68(3):700-10.

Russo J and Russo I H. Cellular basis of breast cancer susceptibility. Oncol Res 1999;11:169-78.

Sanchez-Margalet V, Martin-Romero C. Human leptin signaling in human peripheral blood mononuclear cells: activation of the JAK-STAT pathway. 2001 Cell Immunol 211(1):30-6.

Sleddens H F, Oostra B A, Brinkman A O and Trapman J. Trinucleotide (GGN) repeat polymorphism in the human androgen receptor (AR) gene. Hum.Molec.Genet. 1993;2:493.

Sokoloff P, Riou J F, Martres M P, and Schwartz J C. Presence of dopamine D-2 receptors in human tumoral cell lines. Biochem Biophys Res Commun 1989;162:575-82.

Stoll B A. Western diet, early puberty, and breast cancer risk. Breast Cancer Res Treat 1998;49:187-93.

Szcypka M S, Rainey M A and Palmiter R D. Dopamine in required for hyperphagia n Lep ^{ob/ob} mice. Nature Genetics 2000;25:102-4.

Tavani A, Gallus S, La Vecchia C, Negri E, Montella M, Dal Maso L, and Franceschi S. Risk factors for breast cancer in women under 40 years. Eur J Cancer 1999;35:1361-7.

Tessitore L, Vizio B, Jenkins O, et al. Leptin expression in colorectal and breast cancer patients. 2000 Int J Mol Med 5(4):421-6.

Thompson W D and Janerich D T. Maternal age at birth and risk of breast cancer in daughters. Epidemiology 1990;1:101-6.

Thompson D B, Sutherland J, Apel W, Ossowski V. A physical map at 1p31 encompassing the acute insulin response locus and the leptin receptor. Genomics. 1997 Jan 15;39(2):227-30.

Titus-Ernstoff L, Longnecker M P, Newcomb P A, Dain B, Greenberg E R, Mittendorf R, Stampfer M, and Willett W. Menstrual factors in relation to breast cancer risk. Cancer Epidemiol Biomarkers Prev 1998;7:783-9.

Urbanski H F. Leptin and puberty. 2001 Trends Endocrinol Metab 12(10):428-9. Zarkesh-Esfahani H, Pockley G, Metcalfe R A, et al. High-dose leptin activates human leukocytes via receptor expression on monocytes. 2001 J Immunol 167(8):4593-9.

Zweig M H, Campbell G. Receiver-operating characteristic (ROC) plots: a fundamental evaluation tool in clinical medicine. 1993 Clin Chem 39(4):561-77.

U.S. Patent Application No. 10/319,855.

What is claimed is:

1. A method for determining an individual's susceptibility for having a risk for the development of breast cancer, said method comprising detecting the presence or absence in the individual of a breast cancer risk associated polymorphic allele of a breast cancer risk associated gene selected from the group consisting of: the leptin gene (LEP); the leptin receptor gene (LEPR); and the catechol-O-methyltransferase gene (COMT).

2. A method as in claim 1 wherein said individual's genetic material is analyzed for the presence of a polymorphic allele from one of said genes.

3. A method as in claim 1 which comprises detecting the presence or absence in the individual of at least one breast cancer risk associated allele of a breast cancer risk associated gene selected from the group consisting of:
the LEP dinucleotide repeat polymorphism, wherein risk of development of breast cancer in said individual is high when the LEP genotype is LL and said risk is low in said individual when the LEP genotype is SL or SS;
the LEPR tetranucleotide repeat polymorphism, wherein risk of development of breast cancer in said individual is high when the LEPR genotype is SS or LL and low when the genotype is SL; and
the catechol-O-methyltransferase gene (COMT) Val 108 Met substitution, wherein risk of development of breast cancer in said individual is low when the COMT polymorphism is genotype 1/2 or 2/2 and high when the genotype is 1/1.

4. A method for determining an individual's susceptibility for having a risk for the development of breast cancer, said method comprising detecting the presence or absence in the individual of a breast cancer risk associated polymorphic allele of a breast cancer risk associated gene selected from the group consisting of: the leptin gene (LEP); the leptin receptor gene (LEPR); and the catechol-O-methyltransferase gene (COMT); the dopamine D2 receptor (DRD2) gene; and the androgen receptor (AR) gene.

5. The method of claim 4 which comprises detecting the presence or absence in said individual of one or more alleles from said breast cancer risk associated genes selected from the group consisting of:
the LEP dinucleotide repeat polymorphism, wherein risk of development of breast cancer in said individual is high when the LEP genotype is LL and said risk is low in said individual when the LEP genotype is SL or SS;
the LEPR tetranucleotide repeat polymorphism, wherein risk of development of breast cancer in said individual is high when the LEPR genotype is SS or LL and low when the genotype is SL;
the D2 receptor gene (DRD2) TaqI polymorphism wherein risk of development of breast cancer in said individual is low when the DRD2 polymorphism is genotype 2/2, is intermediate when the genotype is 1/2, and high when the genotype is homozygous for 11;
the catechol-O-methyltransferase gene (COMT) Val 108 Met substitution polymorphism, wherein risk of development of breast cancer in said individual is low when the COMT polymorphism is genotype 1/2 or 2/2 and high when the genotype is 1/1; and;
the Androgen receptor gene (AR) polymorphic trinucleotide repeat sequences, CAG and GGC (GGN), wherein the risk of development of breast cancer in said individual is low when the trinucleotide repeat is LL or SL and risk is high when the trinucleotide repeat is SS.

6. A method for determining an individual's susceptibility for having a risk for the development of breast cancer, said method comprising detecting the presence or absence of a breast cancer risk associated allele from two or more breast cancer risk associated genes selected from the group consisting of the LEP, LEPR, DRD2, COMT and AR genes, and analyzing the additive risks of breast cancer from said breast cancer risk associated genes, wherein risk of development of breast cancer increases according to the number of breast cancer risk associated genes having breast cancer risk associated alleles present in said individual.

7. The method of claim 6 which comprises detecting the presence or absence of a breast cancer risk associated allele for three of said breast cancer risk associated genes.

8. The method of claim 6 which comprises detecting the presence or absence of a breast cancer risk associated allele for four of said breast cancer risk associated genes.

9. The method of claim 6 which comprises detecting the presence or absence of a breast cancer risk associated allele for all of said breast cancer risk associated genes.

10. The method of claim 6, which comprises detecting the presence or absence in said individual of two or more alleles from two or more of said breast cancer risk associated genes selected from the group consisting of:
the LEP dinucleotide repeat polymorphism, wherein risk of development of breast cancer in said individual is high when the LEP genotype is LL and said risk is low in said individual when the LEP genotype is SL or SS;
the LEPR tetranucleotide repeat polymorphism, wherein risk of development of breast cancer in said individual is high when the LEPR genotype is SS or LL and low when the genotype is SL;
the D2 receptor gene (DRD2) TaqI polymorphism wherein risk of development of breast cancer in said individual is low when the DRD2 polymorphism is genotype 2/2, is intermediate when the genotype is 1/2, and high when the genotype is homozygous for 11;
the catechol-O-methyltransferase gene (COMT) Val 108 Met substitution polymorphism, wherein risk of development of breast cancer in said individual is low when the COMT polymorphism is genotype 1/2 or 2/2 and high when the genotype is 1/1; and
the Androgen receptor gene (AR) polymorphic trinucleotide repeat sequences, CAG and GGC (GGN), wherein the risk of development of breast cancer in said individual is low when the trinucleotide repeat is LL or SL and risk is high when the trinucleotide repeat is SS.

11. The method of claim 10 which comprises detecting the presence or absence of a breast cancer risk associated allele for three of said breast cancer risk associated genes.

12. The method of claim 10 which comprises detecting the presence or absence of a breast cancer risk associated allele for four of said breast cancer risk associated genes.

13. The method of claim 10 which comprises detecting the presence or absence of a breast cancer risk associated allele for all of said breast cancer risk associated genes.

14. The method of claim 10 wherein the S allele of the LEP dinucleotide repeat polymorphism is any allele of said repeat that is less than or equal to 207 base pairs and the L allele is any allele of said repeat that is greater than or equal to 208 base pairs.

15. The method of claim 10 wherein the S allele of the LEP dinucleotide repeat polymorphism is any allele of said repeat that is less than or equal to 210 base pairs and the L allele is any allele of said repeat that is greater than or equal to 212 base pairs.

16. A method for determining the specificity, sensitivity and positive and negative likelihood risk of an individual developing breast cancer, the method comprising determining a breast cancer risk score for the individual in an ROC plot, wherein said ROC plot comprises a breast cancer risk score derived from at least two breast cancer risk associated genes selected from the group consisting of LEP, LEPR, DRD2, COMT and AR genes.

17. The method of claim 16 which comprises determining the breast cancer risk score for three of said breast cancer risk associated genes.

18. The method of claim 16 which comprises determining the breast cancer risk score for four of said breast cancer risk associated genes.

19. The method of claim 16 which comprises determining the breast cancer risk score for all of said breast cancer risk associated genes.

20. A method of determining a treatment modality for a human subject suspected of having breast cancer, comprising analyzing the subject's genetic material for the presence or absence of a breast cancer risk associated allele from at least one of the LEP, LEPR, DRD2, COMT and AR genes and determining a treatment on the basis of the presence or absence of one of said risk associated allele or alleles.

21. The method of claim 20, wherein the presence of a breast cancer risk associated allele of two of said genes is determined.

22. The method of claim 20, wherein the presence of a breast cancer risk associated allele of three of said genes is determined.

23. The method of claim 20, wherein the presence of a breast cancer risk associated allele of four of said genes is determined.

24. The method of claim 20, wherein the presence of a breast cancer risk associated allele of all of said genes is determined.

25. A kit suitable for screening a subject to determine whether such subject is at increased risk for having or developing breast cancer associated with the presence of a breast cancer risk allele, said kit comprising:
a) material for determining the subject's genotype with respect to at least one breast cancer-risk associated allele from at least one breast cancer risk associated gene selected from the group consisting of the LEP, LEPR, DRD2, COMT and AR genes;
b) suitable packaging material; and optionally
c) instructional material for use of said kit.

26. A kit as in claim 25 which comprises material for detecting a breast cancer risk allele selected from the group consisting of:
the LEP dinucleotide repeat polymorphism, wherein risk of development of breast cancer in said individual is high when the LEP genotype is LL and said risk is low in said individual when the LEP genotype is SL or SS;
the LEPR tetranucleotide repeat polymorphism, wherein risk of development of breast cancer in said individual is high when the LEPR genotype is SS or LL and low when the genotype is SL;
the D2 receptor gene (DRD2) TaqI polymorphism wherein risk of development of breast cancer in said individual is low when the DRD2 polymorphism is genotype 2/2, is intermediate when the genotype is 1/2, and high when the genotype is homozygous for 11;
the catechol-O-methyltransferase gene (COMT) Val 108 Met substitution polymorphism, wherein risk of development of breast cancer in said individual is low when the COMT polymorphism is genotype 1/2 or 2/2 and high when the genotype is 1/1; and
the Androgen receptor gene (AR) polymorphic trinucleotide repeat sequences, CAG and GGC (GGN), wherein the risk of development of breast cancer in said individual is low when the trinucleotide repeat is LL or SL and risk is high when the trinucleotide repeat is SS.

27. A method for treating a person, diagnosed for having an increased risk for the development of breast cancer, for the prevention of developing said disease, comprising administering to said person an effective amount of an agent counteracting the influence of one or more breast cancer risk associated alleles selected from the group consisting of:
the LEP dinucleotide repeat LL polymorphism;
the LEPR tetranucleotide repeat SS or LL polymorphism;
the catechol-O-methyltransferase gene (COMT) Val 108 Met substitution 1/1 polymorphism;
the D2 receptor gene (DRD2) TaqI 1/1 or ½ polymorphism; and
the Androgen receptor gene (AR) polymorphic trinucleotide repeat sequences, CAG and GGC (GGN) SS polymorphism.


SEQUENCE LISTING (6 sequences)

SEQ ID NO: 1 - DNA, 20 bp, Artificial sequence
  oligonucleotide
gcctaagggaatgagacaca

SEQ ID NO: 2 - DNA, 20 bp, Artificial sequence
  oligonucleotide
atgtgagtttgccaagagct

SEQ ID NO: 3 - DNA, 27 bp, Artificial sequence
  oligonucleotide
ccttcccaacctcctaaagacaacctg

SEQ ID NO: 4 - DNA, 27 bp, Artificial sequence
  oligonucleotide
tgtacagatctgtgctatttttgcagc

SEQ ID NO: 5 - DNA, 21 bp, Artificial sequence
  oligonucleotide
tcgtggacgccgtgattcagg

SEQ ID NO: 6 - DNA, 21 bp, Artificial sequence
  oligonucleotide
aggtctgacaacgggtcaggc